(19) United States
(12) Patent Application Publication
(10) Pub. No.: US 2009/0082657 A1
(43) Pub. Date: Mar. 26, 2009

(54) INTELLIGENT HELIUM COMPRESSOR

(76) Inventors: Michael John Dalchau, Middle Barton (GB); Russell Peter Gore, Abingdon (GB); Rolf Heinrichs, Roettenbach (DE); Trevor Bryan Husband, Banbury (GB); Philip Alan Charles Walton, Witney (GB)

Correspondence Address: SCHIFF HARDIN, LLP;PATENT DEPARTMENT, 6600 SEARS TOWER, CHICAGO, IL, 60606-6473, US

(21) Appl. No.: 12/238,761

(22) Filed: Sep. 26, 2008

(30) Foreign Application Priority Data
Sep. 26, 2007 (GB) 0718703.2

(51) Int. Cl. A61B 5/05; F25B 19/00

(52) U.S. Cl. 600/410; 62/51.1

(57) ABSTRACT

A magnetic resonance imaging system has a superconducting magnet contained within a cryostat, the cryostat being cooled by a cooling system that includes a healing and compressor, a refrigeration device and a local supervisory system. The helium compressor provides compressed helium to the refrigeration device, and the local supervisory system controls operation of the refrigeration device. The helium compressor is in communication with the local supervisory system, and also is able to communicate, independently of the local supervisory system, with a remote service provider.

### BACKGROUND OF THE INVENTION

1. Field of the Invention

The present invention relates to superconducting magnet systems, particularly to refrigeration systems for cooling superconducting magnets. More specifically, the present invention relates to helium compressors provided for operating refrigerators used in cooling superconducting magnets in magnetic resonance imaging (MRI) systems, and especially to apparatus and methods provided to ensure appropriate and effective maintenance of such helium compressors.

2. Description of the Prior Art

In a typical refrigeration arrangement for cooling a superconducting magnet, the superconducting magnet is enclosed within a cryostat, itself typically comprising a cryogen vessel and an outer vacuum chamber which principally serves to provide thermal insulation from ambient temperature. The superconducting magnet is typically cooled to a temperature of approximately 4K by boiling liquid helium, in any one of a number of known alternative arrangements. In order to reduce the consumption of helium, and to reduce the rate of boiling, refrigerators are typically provided, which are able to cool at about 4K, being below the boiling point of helium. This has the effect of recondensing at least some of the boiled-off helium vapor back into liquid form. The provision of such a recondensing refrigerator reduces the consumption of liquid helium, and allows the magnet to be kept cool for a longer time before helium refill is required. Alternatively, or in addition, other refrigerators, cooling to about 10K, may be used to help to maintain the temperature of the superconducting magnet, and to remove heat which has been conducted into the cryostat from ambient.

Such refrigerators, recondensing or not, are typically operated by alternating streams of relatively high-pressure and relatively low-pressure helium. Even the relatively low pressure helium is typically at a pressure in excess of atmospheric pressure, which helps to reduce air ingress to the system. The helium compressor drives high pressure helium gas into a remote cold head unit (refrigerator) in which heat exchange occurs delivering cooling power.

FIG. 1 shows a typical present arrangement of a magnet (not visible) within a cryostat 10, with a mechanical refrigerator 12 providing cooling to the interior of the cryostat. The refrigerator 12 is placed in a helium circuit including high pressure supply line 14, low pressure return line 16 and helium compressor 18.

A magnetic resonance imaging system includes further equipment (not illustrated), such as gradient and field coils, shim coils and a patient table. One or more system electronics cabinet(s) 20 house(s) a magnet supervisory system 22 and other control and measurement equipment 24 which control operation of the magnet, and such further equipment, over communications lines 26. The helium compressor 18 is typically an electromechanical device. It is conventionally mechanically enclosed within the system electronics cabinet(s) 20 but the helium compressor is conventionally a standalone device, in that it is not controlled by any external circuitry, and does not provide signals or information to any external circuitry.

The helium compressor is a hard-working electromechanical device and requires regular servicing to maintain satisfactory operation. Fail-safe devices are typically provided to protect the helium compressor from damage in adverse conditions, and to prevent the compressor from causing damage to other equipment, or personnel. Typically, in response to adverse conditions, or the danger of damage to other equipment, or personnel, one or more of the fail-safe devices will trip, halting the helium compressor. Typically, a visit from a service engineer is required to return the helium compressor to an operating condition. The interval between the helium compressor stopping and it being re-started by a service engineer may be of variable duration, and may risk interrupting the availability of whatever equipment is being cooled by the refrigerator supplied by the helium compressor.

Servicing and diagnosis of the helium compressor and the refrigerator currently require an on-site intervention by a service engineer. This is costly and potentially unnecessary resulting in system downtime and excessive lifecycle costs for the operator.

The fact of the fail-safe devices having tripped, or the helium compressor requiring any other service operation, are typically unknown to the service engineer until summoned by a user. This may result in unnecessary service calls, non optimized service intervals, inadequate service schedules, or unnecessary system down-time.

Furthermore, servicing of helium compressors and associated equipment like refrigerator 12 is generally subject to prescribed service schedules or contracts, which may not be appropriate for specific sites with differing demands, or which may not appropriately deal with the requirements of a helium compressor in its actual operation.

### SUMMARY OF THE INVENTION

An object of the present invention is to provide improved equipment that is capable of reducing the drawbacks of the present arrangements as described above. In particular, the invention seeks to allow a remote service provider to be accurately and rapidly informed of fail-safe devices being tripped, or the helium compressor requiring any other service operation. Furthermore, the present invention aims to enable the remote service provider to perform certain service and diagnostic procedures remotely by remotely supplying service commands to the helium compressor.

The above object is achieved in accordance with the present invention in a magnetic resonance imaging system having a superconducting magnet contained within a cryostat, the cryostat being cooled by a cooling system that includes a helium compressor and a refrigeration device, which are supervised by a local supervisory system. The helium compressor provides compressed helium to the refrigeration device. The local supervisory system controls operation of the refrigeration device and the helium compressor. The helium compressor is in communication with the local supervisory system and the helium compressor is able to communicate with a remote service provider, independently of the local supervisory system.

### BRIEF DESCRIPTION OF THE DRAWINGS

FIG. 1, as described above, illustrates a known arrangement comprising a helium compressor in a system further comprising a refrigerator supplied by the helium compressor.

FIG. 2 illustrates an arrangement according to the present invention having a helium compressor in a system further having a refrigerator supplied by the helium compressor.

### DESCRIPTION OF THE PREFERRED EMBODIMENTS

The present invention provides remote diagnostic and service capability to a helium compressor, such that the helium compressor includes a communications device allowing it to signal service requests to a remote service provider, and to receive service commands from a remote service provider, independently of a local supervisory system.

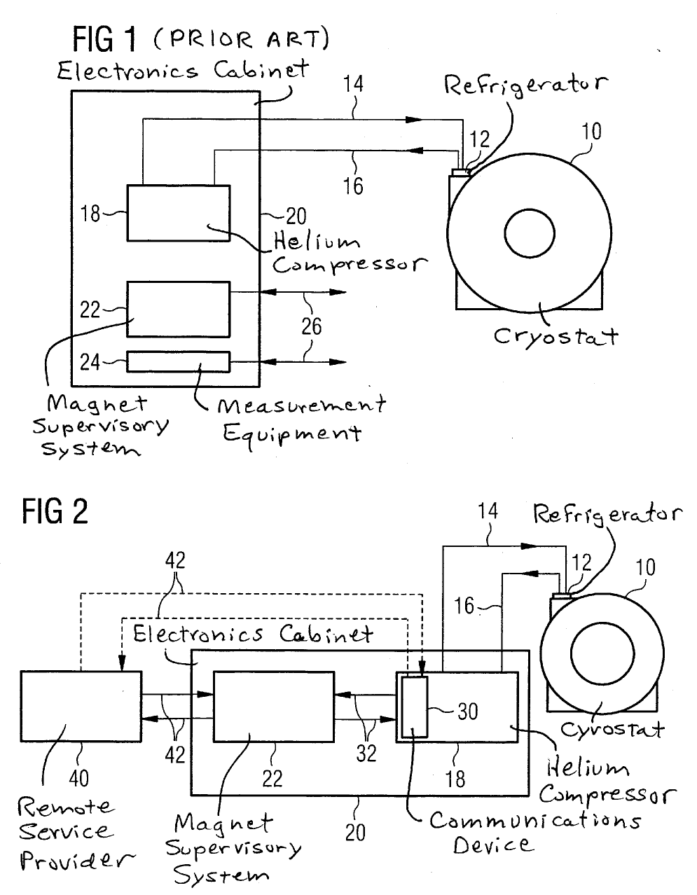

FIG. 2 illustrates an arrangement according to the present invention comprising a helium compressor in a system further comprising a refrigerator supplied by the helium compressor. In FIG. 2, features common with those shown in FIG. 1 carry common reference numerals.

According to an aspect of the present invention, helium compressor 18 is provided with a communications device 30 which enables it to communicate with magnet supervisory system 22 along local communications paths 32, and/or to communicate with a remote service provider 40 along telecommunications paths 42. Local communications paths 32 may preferably be provided by wired connections between the helium compressor 18 and the magnet supervisory system 22, although alternative connection arrangements may of course be provided such as short-range radio links like BLUETOOTH®, or infra-red links, inductively coupled transmitter and receiver and so on. The telecommunications links 42 are preferably provided over the Internet, but may be provided by any known telecommunications link suitable for reaching the remote service provider 40. Examples may include telephone lines for fax, synthetic voice, or other standards; dedicated or other radio links.

Regarding the local communications paths 32 between the helium compressor 18 and the magnet supervisory system 22, CANBUS technology has been found appropriate. CANBUS is a standard for transmitting brief messages over short distances, and has become popular in automotive applications. CANopen is a derivative communications profile, standardized to EN50325, and has been found to be particularly suitable over wired connections in the present invention. Other standard short-range communication protocols such as RS232, RS422 and so on could be used as alternatives.

The provision of communications device 30 in the compressor 18 enables data relating to historical and current operational performance and conditions to be collected and transferred to magnet supervisory system 22 and/or to remote service provider 40. This communications arrangement enables direct connection between the helium compressor 18 and the magnet supervisory system 22, as well as with a remote service provider 40. The magnet supervisory system 22 may also communicate with remote service provider 40 over telecommunications paths 42.

In an exemplary scenario, a fail-safe device has tripped, causing the helium compressor to stop, as described above. The fact of the fail-safe tripping may immediately be reported to the magnet supervisory system 22, and/or remote service provider 40. If the indicated problem is easily solved, for example, an equipment cover being open, or a person being present in a hazardous position, a user may be prompted by the magnet supervisory system 22 to remove the cause of the fail-safe trip, to reset the fail-safe device if necessary, and re-start the helium compressor 18. In this way, a costly site visit by a service engineer may be avoided. Moreover, a more serious fail-safe trip, such as contamination being detected in one of the helium lines 14, 16 may be indicated directly to the remote service provider 40, who may schedule a site visit by a service engineer even before the user is aware of the problem.

Another advantage of the present invention is that remote diagnostics would be possible. This would allow a service engineer at the remote service provider 40 to interrogate the helium compressor before traveling to site, for example from a central service location over wired networks or over a wireless internet device such as a WAP enabled mobile telephone or a portable computer using WiFi or mobile telephone networks, for example from the engineer's vehicle. This provides advantages in that site service visits may be provided sooner than with conventional arrangements, and the service engineer has a better idea of the work required before reaching site, so the engineer may be better prepared to return the helium compressor to operation, resulting in reduced down-time. The engineer may determine that a simple service function, such as a reset operation, may be performed by the user, and may instruct the user accordingly by telephone, fax, email or a dedicated messaging service built into the arrangement of FIG. 2. Service operations may be scheduled and tailored according to the demands of each system.

According to another aspect of the present invention, the helium compressor 18 is arranged such that the included communications device 30 receives service commands from the remote service provider 40, and is capable of performing a range of control functions. Minor remote control functions may accordingly be performed, as controlled by the service engineer.

Taking the particular example of an MRI system, it is common practice for the magnet supervisory system 22 to be in a “standby” or other inoperative condition when the MRI system is not in use for imaging or for servicing operations. The helium compressor 18, on the other hand, is typically continuously active, to provide effective helium compression, refrigeration and cooling to the magnet on a permanent basis, to ensure that the cooled magnet is available for use when required. As the magnet supervisory system 22 is therefore unavailable for certain periods of time, it may be important for the helium compressor to be able to communicate directly with the remote service centre 40 to obtain servicing or to report faults even when the magnet supervisory system 22 is unavailable.

The present invention is accordingly believed to result in improved availability of the magnet system, by reducing the need for site service visits, and by ensuring that the site service visits which become necessary are effective. This has not always been the case in the past. Typically, one manufacturer would make a stand-alone helium compressor and would specify a recommended service schedule. A manufacturer of MRI systems may then specify the use of such a helium compressor, but would take no involvement in the maintenance or servicing of the compressor. A service provider would make site visits according to a service schedule, or when called by a user, but would have no direct access to the helium compressor in order to perform remote diagnostics or remote servicing.

The present invention improves upon this conventional arrangement by providing intercommunication and control functions between a helium compressor and a local supervisory system and a remote service provider. This enables improved service arrangements, reduced system down-time and avoids the delays and expense of unnecessary site visits, by providing an intercommunicating system involving the MRI system, the helium compressor and the remote service provider.

Communication between the helium compressor 18 and supervisory system 22 and/or remote service provider 40 provides at least the following advantages:
Remote diagnostics of the refrigeration system are possible.
Remote operation can be performed (this allows a remote service engineer to test the refrigeration system before traveling to site).
Remote service calls is enabled (where the helium compressor calls the service centre when a fault is detected).
Parameters within the helium compressor can be logged.

Furthermore, while the present invention has been described with particular reference to embodiments in which a communications device is provided within the helium compressor, other arrangements performing the same function fall within the scope of the present invention. For example, in certain embodiments, the magnet supervisory system and the communications device are integrated and are located on the cryostat. In other embodiments, the magnet supervisory system and the communications device are integrated and are located within the helium compressor. The present invention accordingly encompasses all arrangements in which the helium compressor is connected so as to communicate with the local supervisory system and is connected so as to communicate, independently of the local supervisory system, with a remote service provider.

We claim as our invention:

1. A magnetic resonance imaging system comprising a superconducting magnet housed within a cryostat, the cryostat being cooled by a cooling system comprising a helium compressor, a refrigeration device and a local supervisory system, the helium compressor providing compressed helium to the refrigeration device, a local supervisory system configured to control operation of the refrigeration device, and said helium compressor being in communication with the local supervisory system and being able to communicate, independently of the local supervisory system, with a remote service provider.

2. A magnetic resonance imaging system according to claim 1 wherein the helium compressor includes a communications device for communication with the remote service provider, that allows the helium compressor to communicate with the remote service provider to provide information, to signal service requests to the remote service provider, and to receive service commands from the remote service provider.

3. A magnetic resonance imaging system according to claim 1 wherein the helium compressor includes a communications device for communication with the local supervisory system, that allows the helium compressor to communicate with the local supervisory system to provide information, to signal service requests to the local supervisory system, and receive service commands from the local supervisory system.

4. A magnetic resonance imaging system according to claim 1 wherein the local supervisory system includes a communications device for communication with the helium compressor, that allows the helium compressor to provide information, to signal service requests to the local supervisory system, and receive service commands from the local supervisory system.

5. A magnetic resonance imaging system according to claim 1 wherein the local supervisory system is configured to communicate with a remote service provider, to provide information, to pass signal service requests from the helium compressor to the remote service provider, and to pass service commands from the remote service provider to the helium compressor.

6. A magnetic resonance imaging system according to claim 1 wherein the local supervisory system includes a communications device for communication with the helium compressor, that allows the helium compressor to signal service requests to the local supervisory system, and to receive service commands from the local supervisory system, and the local supervisory system and the communications device are integrated and are located on the cryostat.
